(19) United States
(12) Patent Application Publication
(10) Pub. No.: US 2024/0161276 A1
(43) Pub. Date: May 16, 2024

(54) SYSTEMS AND METHODS FOR PREDICTING RESPONSE OF TRIPLE-NEGATIVE BREAST CANCER TO NEOADJUVANT CHEMOTHERAPY

(71) Applicants: PATHOMIQ INC., Saratoga, CA (US); Board of Regents, The University of Texas System, Austin, TX (US)

(72) Inventors: Parag Jain, Palo Alto, CA (US); Rajat Roy, Saratoga, CA (US); Savitri Krishnamurthy, Houston, TX (US); Debasish Tripathy, Houston, TX (US)

(21) Appl. No.: 17/988,600

(22) Filed: Nov. 16, 2022

(51) Int. Cl. G06T 7/00; G06T 7/11; G06V 20/69; G16H 30/40; G16H 50/20

(52) U.S. Cl. CPC G06T 7/0012; G06T 7/11; G06V 20/698; G16H 30/40; G16H 50/20; G06T 2207/10056; G06T 2207/20081; G06T 2207/20084; G06T 2207/30024

(57) ABSTRACT

Disclosed are systems and methods for predicting response of triple-negative breast cancer to neoadjuvant chemotherapy using a deep convolutional neural network-based artificial intelligence tool a method of predicting patient response to therapy. The system divides patient tissue image slides into multiple tiles. A convolutional neural network (CNN) is trained based on the multiple tiles. The system may perform artifact detection and cancer classification to identify patterns and features that capture tumor cell heterogeneity along with stromal and tumor micro environment (TME) components of a second set of patient tissue image tiles.

### FIELD

This invention relates generally to the field of disease detection and treatment identification, and in particular to using artificial intelligence in predicting response of triple-negative breast cancer to neoadjuvant chemotherapy.

### BACKGROUND

Current methods of developing treatment options for triple-negative breast cancer (TNBC) patients in a neoadjuvant setting are based on the stage of disease in the patient. The Standard-of-Care therapy today is to offer a combination of chemotherapy regimens followed by surgery. It is determined at the time of surgery whether the patient had pathological Complete Response (pCR), or had Partial or No Response (PR/NR). 30-40% of TNBC patients achieve pCR, and generally tend to have good outcomes in terms of Progression Free Survival (PFS) and Overall Survival (OS). Oncologists today are unable to identify upfront which patient will have PR/NR, and could benefit from advanced/experimental therapy at an early stage. Pharmaceutical companies are running clinical trial to get advanced therapy to these patients, but most trials fail to attain statistical significance as patients are randomly selected without knowing who will achieve pCR with chemotherapy alone, and who will actually benefit from advanced therapy. Systems and methods that can identify who will achieve pCR vs PR/NR to neoadjuvant therapy for TNBC patients are warranted to select the right treatment for the right patient.

Current techniques to discover biomarkers that predict TNBC chemotherapy response are focused on structured molecular data, such as genomic and proteomic data, and have limited success. The molecular analysis is done at a whole tissue level which delivers an average molecular signature across 10s of 1000s of cancer, benign and micro-environmental (stroma, immune etc.) cells. This technique works when a single or few genes are heavily overexpressed in cancer and/or its micro-environment. However, tumors are inherently heterogeneous and there are several molecular subtypes with varying levels of expression in a tissue sample. In many cases, <1% of the cancer cells may be the most aggressive and informative of molecular pathways of the disease and of the patient outcome. This molecular signal gets lost when averaged over the entire tissue.

Also, tumor alone does not capture the full picture—it is the spatial interaction of the tumor with the Tumor Micro-Environment (TME) that includes stroma, several types of immune cells, blood vessels etc. whose interplay determines tumor aggressiveness and patient response to a particular therapy. Current proteogenomic analysis is not able to capture the TME dynamics, nor is there one single RNA or protein that is driving patient response and outcome.

Histopathology as observed through Hematoxylin and Eosin (H & E) stained slides is currently used for TNBC diagnosis, and these slides are routinely collected in clinical practice. However, there are distinct morphological changes in the way the tumor cells organize themselves and these patterns evolve as the cells undergo molecular changes. These molecular changes such as gene mutations, copy number variations, gene fusions etc. in many cases result in morphological changes. However, it is not known what these changes are, and hence pathologists in standard-of-care are unable to discern which pattern or patterns are indicative of such changes. These images also capture distinct tumor micro-environments with spatial organization of the tumor, immune, stromal and other cells which influence patient response to therapy.

Traditional supervised AI approaches are focused on the detection of “known” biomarkers, such as tumor detection, counting TILs, PD-L1 quantification etc. The accuracy of these models is limited to the predictive power of features that are already known, and the major contribution is automation and inter-observer consistency.

Consequently, there is a need for computer aided systems and methods that can discover, cluster and extract novel morphometric features that correlate with TNBC patient outcome from histopathology slides in an unsupervised manner. The methods also identify specific Regions of Interest (ROIs) on the tissue slides that are most predictive and does molecular analysis on the ROIs to discover novel biomarkers that predict patient response and integrate to the platform to further improve predictive response.

### SUMMARY

In contrast to previous systems and methods, the systems and methods described herein provides a novel and previously “unknown” morphological features on digitized images of H & E-stained slides that drive tumor progression. The system converts image patches into mathematical vector representations to generate hundreds of clusters of morphologically similar patterns using state-of-the-art Deep Convolutional Neural Network (CNN)-based models. It then ranks these image clusters to identify novel Regions of Interest (ROIs) on the H & E-stained slides which have high or low prognostic/predictive values correlated to patient disease outcome information to generate a morphometric score for therapy response prediction. These ROIs capture both cancer as well as TME landscapes, including spatial distribution of the immune and stromal cells relative to the cancer zones that play major roles in tumor progression and resistance to therapy.

In one aspect, predicting response of triple-negative breast cancer to neoadjuvant chemotherapy using a deep convolutional neural network-based artificial intelligence tool a method of predicting patient response to therapy is disclosed.

In one embodiment, the a method for predicting patient outcome to neoadjuvant chemotherapy for triple negative breast cancer, includes the process of: dividing a first set of patient tissue image slides into a plurality of tiles of a predetermined pixel size; training a convolution neural network using randomly sampled tiles of 64 pixels by 64 pixels at 20 times resolution of a sample, 256 pixels by 256 pixels at 20 times the resolution of the sample, and 1024 pixels by 1024 pixels at 5 times the resolution of the sample; transforming the plurality of tiles into a high-dimensional vector representation such that vectors of morphologically similar patterns cluster together; using the trained neural network, performing artifact detection and cancer classification to identify patterns and features that capture tumor cell heterogeneity along with stromal and tumor micro environment (TME) components of a second set of patient tissue image tiles; and performing feature ranking on the second set of patient tissue image tiles.

### BRIEF DESCRIPTION OF THE DRAWINGS

These drawings and the associated description herein are provided to illustrate specific embodiments of the invention and are not intended to be limiting.

FIG. 1 illustrates an example of disease progression stages in relation to available treatment options.

FIG. 2 illustrates an exemplary process of a workflow for predicting patient outcome to neoadjuvant chemotherapy for triple negative breast cancer.

FIG. 3 illustrates an exemplary feature extraction module according to an embodiment.

FIG. 4 illustrates an example of an underlying semantic structure of histopathology images.

FIG. 5 illustrates an example feature ranking process according to an embodiment.

FIG. 6 illustrates a table and graph depicting a response to neoadjuvant chemotherapy.

FIG. 7 illustrates an example heatmap relating to ROIs for PR/NR according to an embodiment.

FIG. 8 illustrates an example heatmap relating to ROIs for pCR.

FIG. 9 illustrates a method 900 for predicting patient outcome to neoadjuvant chemotherapy for triple negative breast cancer.

FIG. 10 illustrates a table depicting patient demographics and characteristics.

FIG. 11 illustrates a table depicting pathological findings.

FIG. 12 illustrates and example of tiling an invasive tumor, stroma, necrosis and stromal tumor infiltrating lymphocytes.

FIG. 13 illustrates exemplary AI prediction scores for four charts A, B, C and D.

FIG. 14 illustrates an exemplary graph depicting quartile analysis.

FIG. 15 illustrates exemplary images from pre-chemotherapy biopsy and final surgical pathology.

FIG. 16 is a diagram illustrating an exemplary computer that may perform processing in some embodiments.

### DETAILED DESCRIPTION

The following detailed description of certain embodiments presents various descriptions of specific embodiments of the invention. However, the invention can be embodied in a multitude of different ways as defined and covered by the claims.

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

When the terms “one”, “a” or “an” are used in the disclosure, they mean “at least one” or “one or more”, unless otherwise indicated.

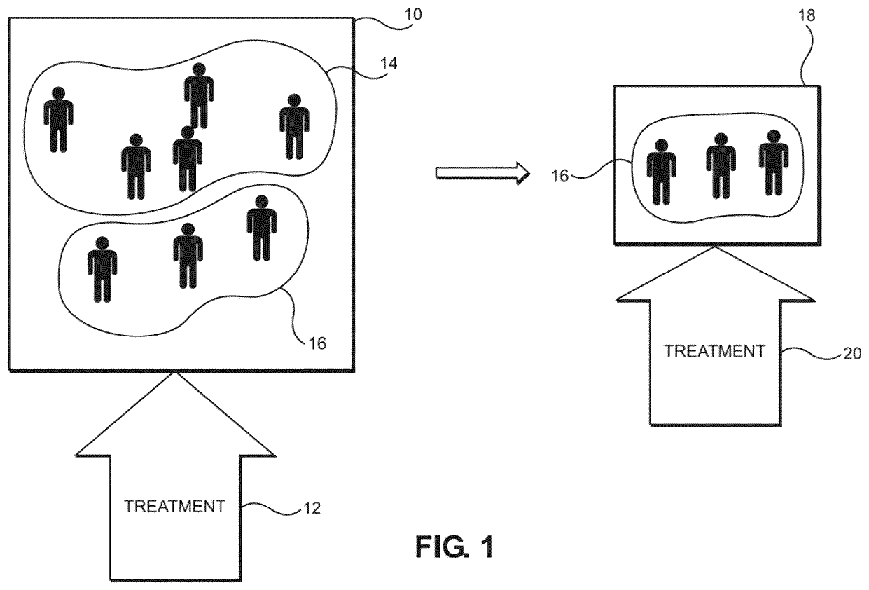

In the context of disease treatment, current medical practice and standard-of-care (SOC) might treat patients based on stages of a disease and the patient's responsiveness to available treatments for those stages. These treatments in case of cancer may include administrating drugs, radiation therapy, surgery or other forms of treatment. If the patient does not respond to available treatments for the stage of disease the patient is in, the patient transitions to a different stage, where different, and potentially more aggressive treatment options may be applied. Later stage treatment options may include experimental or advanced therapy options. FIG. 1 illustrates an example of disease progression stages in relation to available treatment options. In the first disease stage 10, treatment options 12 are applied. A first group of patients 14 respond well to the treatment options 12. A second group of patients 16 do not respond well to the treatment options 12 and their disease progresses to the disease stage 18. Treatment options 20 are available and may be applied for the second group patients 16 in the disease stage 18.

The dynamics of disease progression and applied treatment, in some cases, are as follows. Most patients in earlier stages, respond well to treatment, but a smaller percentage of patients in earlier stages do not respond well to the treatment options applied in those earlier stages. For example, the first group of patients 14 that respond well to treatment options 12 can be a substantially larger number than the second patient group 16 that do not respond well to the treatment options 12. Furthermore, the treatment options that are available to be applied in the later stages can have more efficacy if they are applied in earlier stages. In other words, for the second patient group 16, if treatment options 20, were applied, when those patients were in an earlier disease stage 10, the treatment options 20 may have had more efficacy.

Furthermore, later stage treatment options 20 can include potentially more aggressive treatments or experimental advance therapy options. In some cases, the late stage treatment options 20 can be experimental in nature and can include treatment options that governmental approval may not yet have been obtained. Nonetheless, the second group patients 16 may substantially benefit from those treatments if they were applied in an earlier disease stage 10. Consequently, in terms of disease treatment efficacy and treatment discovery, systems and methods that help early identification of patients that respond well to available treatment options can be beneficial and needed.

Also, pharmaceutical companies are running several thousand clinical trials to get advanced and novel drugs to market across several cancer indications. Lack of reliable predictive biomarkers to identify responders vs non-responders to these drugs result in random selection of patients for the trials and contributes to the low success rate. Even when some of these trials succeed, only a small percentage of patients respond to the drugs when administered in clinical practice. Consequently, there is a huge unmet need for pharmaceutical companies to identify responders to a new drug early on.

Biochemical signatures, biomarkers etc. can be used to predict patient outcome. Some genomics and proteomics techniques to discover biomarkers that predict patient response are focused on biochemical markers and the structured molecular data of those biochemical markers, such as DNA, RNA, and protein data. This approach has major challenges. First, molecular analysis is done from DNA, RNA, and protein extracted from whole tissue which delivers an average molecular signature across tens of thousands of cancer, benign and micro-environmental (e.g., stroma, immune etc.) cells. Consequently, this approach works better when a single or few genes are heavily overexpressed or under-expressed across an entire tissue. However, tumors can be inherently heterogeneous, and there are several molecular subtypes with varying levels of aggressiveness that show up in the same tumor and the tumor micro-environment. This molecular signal gets lost when averaged over an entire tissue.

Second, biochemical, molecular, structural or other analysis of tumor alone do not present a full picture of the disease. In many cases, it is the spatial interaction of the tumor with the tumor micro-environment (TME), including the stroma, several types of immune cells, blood vessels etc., whose interplay determines patient response. Many current genomic analyses are not able to capture the TME dynamics, nor is there one single RNA or protein that can be linked to driving patient response. Nevertheless, Histopathology remains the cornerstone of cancer diagnosis. Many molecular changes and TME elements that are linked to disease can result in morphological changes that are visible on tissue slides. Consequently, systems and methods that can identify and extract morphometric features that correlate with patient outcome from histopathology slides are valuable in disease treatment. Therefore, it is advantageous to employ artificial intelligence (AI) in an unsupervised manner to identify and extract these morphologic features.

Furthermore, the field of studying biomarkers and identification of morphologic features for drug and treatment discovery can be slowed down by the sheer number of samples and patient data that need to be analyzed to identify biomarkers of interest. For example, some methods rely on or work in conjunction with laboratory test results. Described embodiments substantially reduce the volume and number of data that need to be analyzed in a laboratory environment, making the applications of the described embodiments more practical than existing systems. For example, the described embodiments can identify regions of interest (ROIs) on tissue slides that are more predictive, and more promising or relevant for performing laboratory molecular analysis to identify predictive biomarkers of patient response. The identification of aberrant genes/proteins present in the ROI known to be involved in therapy response prediction may also enhance easy detection of disease or therapy response biomarkers, unlike techniques which operate on the whole tissue slide, where the abnormality could be masked by the large preponderance of cells with normal proteogenomic patterns.

Current methods of cancer diagnosis, and in some cases cancer prognosis using histopathology include trained pathologists examining sample slides from a patient. The pathologists examine patient cells and look for patterns and other markers as identified in one or more SOC trade guidelines, such as guidelines published by the national comprehensive cancer network (NCCN). Pathologists identify type of cells, they are observing in the sample, as well as identifying whether a patient sample contains benign or malignant tumor cells, and in some cases, a grading of the detected cancer cells. The SOC guidelines are typically generated by researchers and health care professionals who through their years of experience observing patient samples have accumulated a knowledge-base of correlations between features in patient sample tissue and cases of other patients in the past and an associated outcome with the observed features or combination of several specific features. In this paradigm, the identification of biomarkers is limited to the guidelines and past experiences of the healthcare professionals. The process of updating the guidelines and the way the pathologists scan, examine and identify biomarkers is therefore a dynamic and at the same time a slow process.

In other words, the current methodologies of biomarker identification can include matching features from a sample space against a limited-scope database of known biomarkers. The described embodiments, on the other hand, can utilize unsupervised artificial intelligence architectures to scan tissue sample image data at a much faster speed and also identify biomarkers predictive of patient outcome that has never been previously identified.

Another challenge with traditional methods of identification of biomarkers and drug target is that diseases, such as cancer can be highly heterogenous and evolving over time. One tumor may include many different molecular subtypes some of which may be biomarkers predictive of patient response. Many techniques look at a small subset of potential molecular subtypes by analyzing a whole tissue slide from a patient. That approach has identified some useful biomarkers, but a wealth of data and information in each patient slide also remains unexamined. As a result, many patients still get baseline treatments, even though they may be good candidates for a different treatment option. Not knowing the relevant biomarkers, the success rate of many treatments is lower than maximum because a large patient population are treated with the same treatment options, without regards to the anticipated response. What is worse, is that in the absence of better alternative, low-success rate treatment options become SOC. Systems and methods that can identify biomarkers predictive of patient response will help to identify patients, who are good candidates for a specific treatment option and deliver targeted and personalized therapies to an individual patient.

Furthermore, patient outcome and responsiveness can be a multimodal problem, where tumor alone or normal disease pathways and mechanisms may not be the only relevant factors. For example, a tumor micro-environment (TME) can play a significant role in patient responsiveness. A drug can be correctly designed based on a disease or tumor, but it might not reach the correct target in the patient if the drug is not designed with the TME of the tumor cells in mind. As an example, a drug might be correctly designed to activate immune system, but in some patients, the tumor might have few infiltrated immune cells, or might have immune suppressor cells nullifying the drug effect. The described embodiments use, the TME of a cell, including stroma, immune cells, blood vessels etc., as well as the tumor cells, when identifying biomarkers, thus enabling the selection of treatment options with higher success potential.

In one sense, the traditional methods of biomarker identification have relied on molecular biologists and pathologists as the initial actors that identified the biomarkers. The results of human-driven identification of biomarkers predictive of patient response were then verified using bioinformatics and statistical analysis. As discussed earlier, the human-driven method of biomarker identification is necessarily limited in the size and number of patient samples capable of being analyzed in laboratory settings, and by the patterns and structures that have previously been identified in research and trade guidelines. The disclosed embodiments, on the other hand, analyze tissue samples in a patient or patient population and identify biomarkers predictive of patient response that may not have been previously known. The results, including the newly identified biomarkers, can be further confirmed by pathologists or biologists in a laboratory setting.

For pharma and oncologists, each stage and sub-type of disease and each potential drug is a unique challenge for biomarker discovery and drug development. The disclosed computer aided systems and methods that correlate disease outcome with tissue morphology are agnostic to the type of cancer and of its treatment. The systems and methods rank morphological features based on known patient outcome to a particular drug to treat a specific disease but do not depend upon the drug mechanism itself. They can therefore be applied to any disease such as cancer that changes morphology and the treatment of interest.

In another embodiment, the method can also be used to identify morphometric features on patient tissues that correlate with a particular molecular change, such as protein loss or gene mutations without the need for a molecular test such as immunohistochemistry (IHC) or gene panel testing. It can rank the morphological features in an unsupervised manner using only the molecular status as label and determine the lead morphology features that can be related to the molecular change.

### Predicting Patient Outcome to Neoadjuvant Chemotherapy for Triple Negative Breast Cancer

The following discussion relates generally to the field of disease detection and treatment identification, and in particular to using artificial intelligence in predicting patient outcome to neoadjuvant chemotherapy treatment for Triple Negative Breast Cancer (TNBC) patients.

Triple-negative breast cancer (TNBC) is an aggressive variant of invasive mammary tumor that accounts for 15-20% of all breast tumors and is associated with high recurrence rates and poor overall survival. By definition, TNBC is characterized by the absence of expression of estrogen receptor (ER), progesterone receptor (PR), and human epidermal growth factor receptor 2 (HER2), which are the recognized targets for tailored treatment of breast tumors. Neoadjuvant chemotherapy (NAC) including anthracycline and taxanes remain the main treatment option for patients diagnosed with early and advanced stages of TNBC. Pathologic complete response (pCR) with no evidence of residual tumor in the breast and locoregional lymph nodes is achieved in approximately 30-40% of patients with early-stage TNBC following treatment with NAC. Achieving pCR to NAC is well established as a surrogate marker and predictor of 5-year disease-free and overall survival in patients with TNBC. The possibility of predicting response to NAC at the time of initial diagnosis before the initiation of therapy rather than after the completion of therapy would be an important pre-requisite to prospective trials testing escalated or de-escalated regimens based on the probability of response or resistance to NAC. For example, prediction of pCR to NAC with reliable, reproducible, and validated indicators may lead to consideration of avoiding toxic chemotherapeutic agents such as anthracyclines. Further, prediction of ineffectiveness of NAC with a high probability of encountering residual disease may provide the opportunity to alter the treatment by using chemotherapeutic agents potentially coupled with biologically targeted agents. Therefore, there is compelling interest in identifying robust predictors of response to NAC to guide clinical management of TNBC.

In recent years, the advent of whole-slide scanners for digitizing hematoxylin and eosin (H & E) stained glass slides has revolutionized pathology practice. The light microscope introduced more than 100 years back for conventional histopathological examination is becoming less of a necessity with the availability of whole slide images (WSIs) for digital viewing and interpretation. The availability of WSIs of H & E-stained tissue, together with the rapidly advancing technology of computational approaches, brings unprecedented opportunity for pathology analyses. There is increasing interest in using artificial intelligence (AI) enabled tools encompassing machine learning and deep learning (DL) approaches for several applications related to digital pathology. DL approaches are becoming increasingly popular AI tools for interrogating WSIs because they do not rely on hand crafted features and can learn representations directly from the primary data. This approach has been widely used for several diagnostic tasks in digital pathology, such as for identification and quantification of cells and for delineation of histological features or areas of interest in WSIs. In addition to CNN-based AI analyses' potential for diagnostic applications, they can also be powerful tools for extracting the hidden sub visual features of the tumor and stroma that can be exploited for making predictions regarding the behavior of malignant tumors. A tumor's morphological appearance in H & E-stained tissue sections is essentially a manifestation of the tumor's composite result of the global genomic and proteomic expression of the tumor. Automated AI-based extraction of the features in H & E stained WSIs enables the evaluation of multiple sub visual morphometric features. Extraction of the features demonstrated by tumor and stroma can effectively capture the complexity of cell signaling, cellular interactions, and the tumor microenvironment (TME). Therefore, AI-based approaches can be used to obtain unique information about tumors that was hitherto not recognized. The ability to extract novel morphometric features from H & E-stained tissue sections beyond what can be recognized by conventional histopathological examination renders AI enabled tools an unmatched power to explore their potential as prognostic and predictive tools. The purpose of this study was to develop and validate a novel deep CNN-based AI-based prediction system built by a team of computer scientists and pathologists using features of the invasive tumor and stroma in H & E stained WSIs of core biopsies for predicting response of TNBC patients to NAC.

Triple-negative breast cancer (TNBC) is commonly treated with neoadjuvant chemotherapy (NAC). Ability to predict response to NAC from pretreatment core biopsy of the tumor can improve personalization of treatment. We developed and validated a deep convolutional neural network (CNN)—based artificial intelligence (AI) model to extract sub visual morphometric features of TNBC to predict response to NAC.

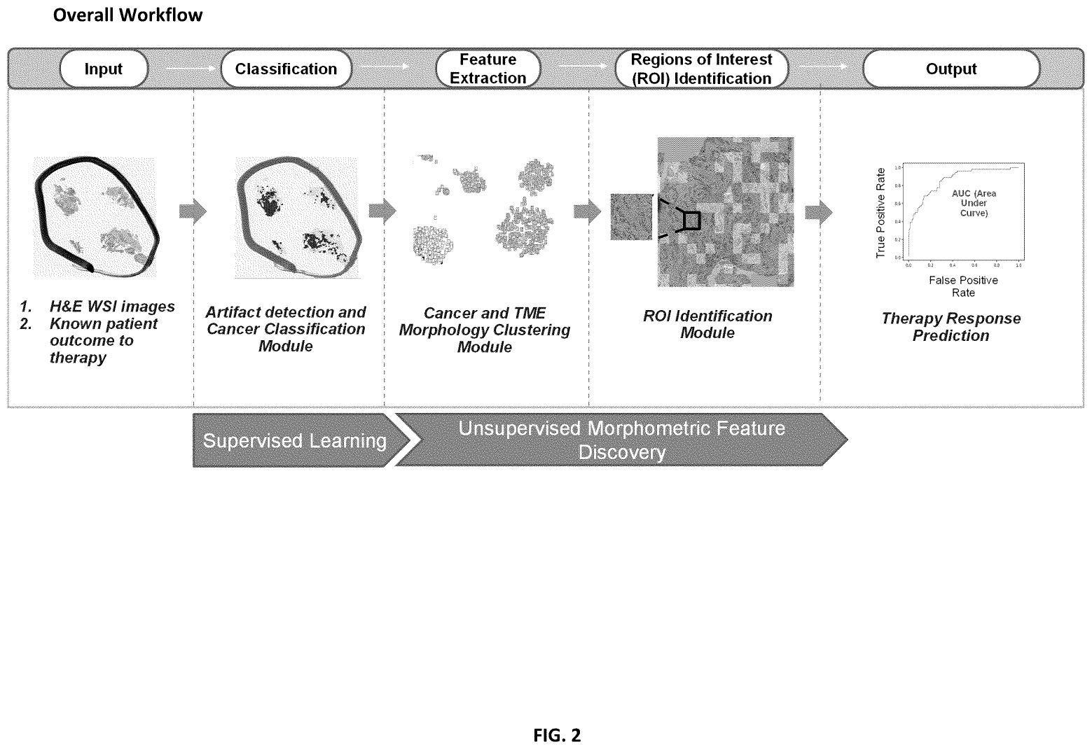

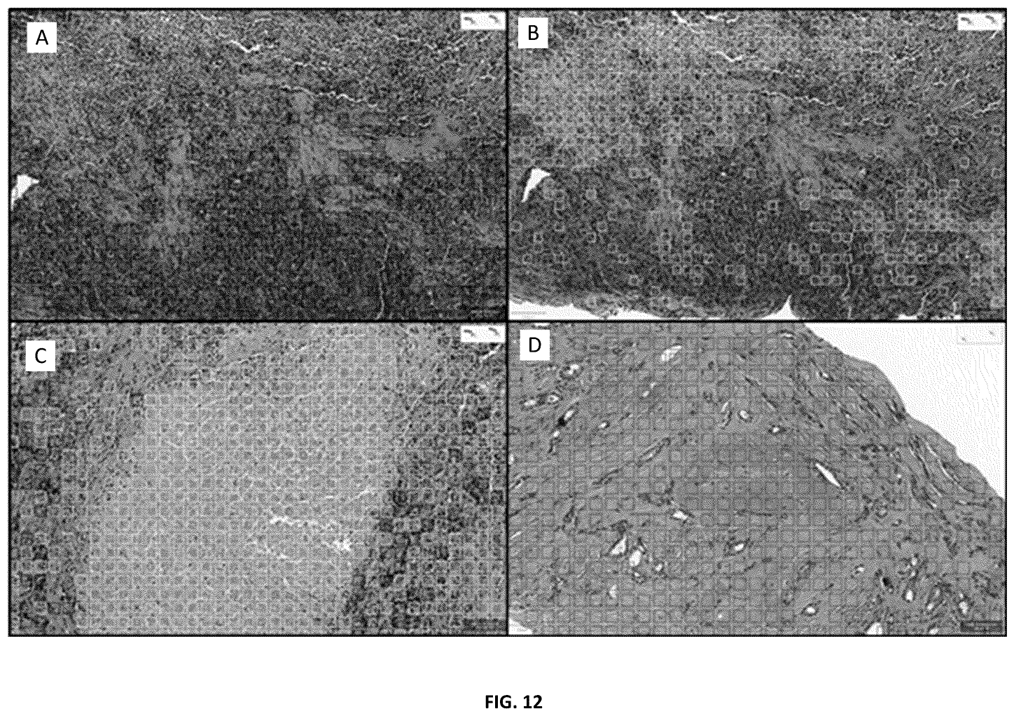

Referring now to FIGS. 2-16, further examples for predicting patient outcome to neoadjuvant chemotherapy for triple negative breast cancer is described. The following process ad described below may be utilized with the systems and methods as previously described herein.

FIG. 2 illustrates an exemplary process of a workflow for predicting patient outcome to neoadjuvant chemotherapy for triple negative breast cancer. In one embodiment, the workflow includes multiple operations. In a first step, the system receives input of H & E WSI images and known patient outcome to therapy. In a next step, the system performs classification by an artifact detection and cancer classification module. In a next step, the system performs feature extraction by a cancer and TME clustering module. In a next step, the system performs Region of Interest (ROI) identification via a ROI identification module. In the last step, the system determines an output of a therapy response prediction.

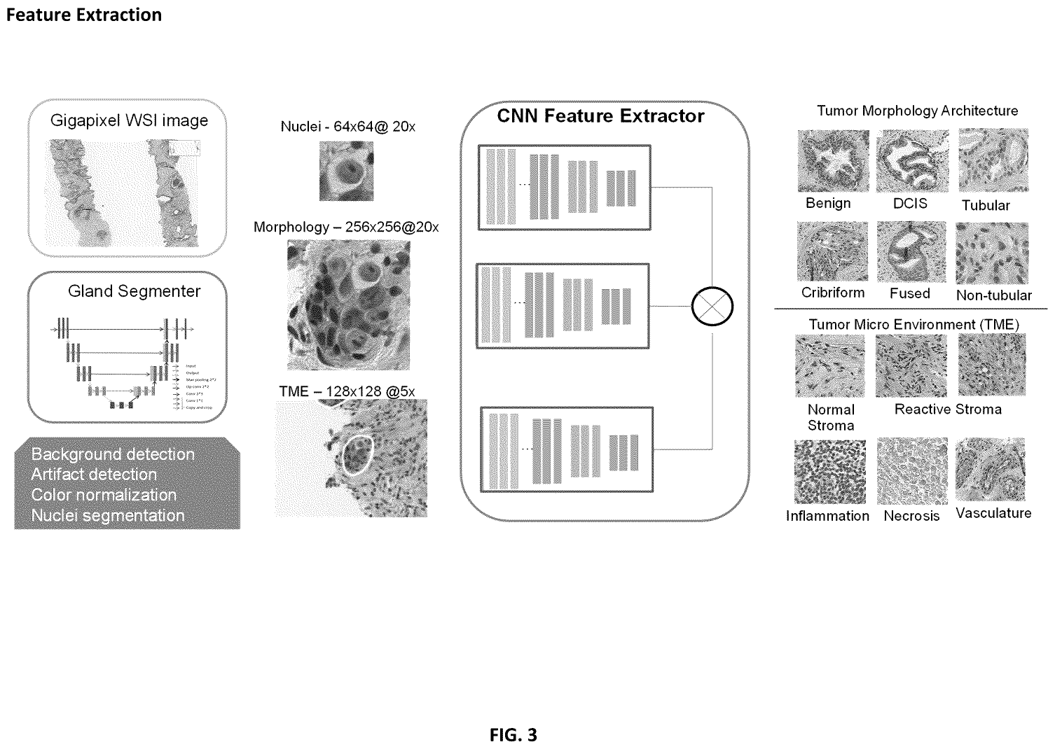

FIG. 3 illustrates an exemplary feature extraction module according to an embodiment. The system may perform feature extraction of images and process them via a CNN feature extractor.

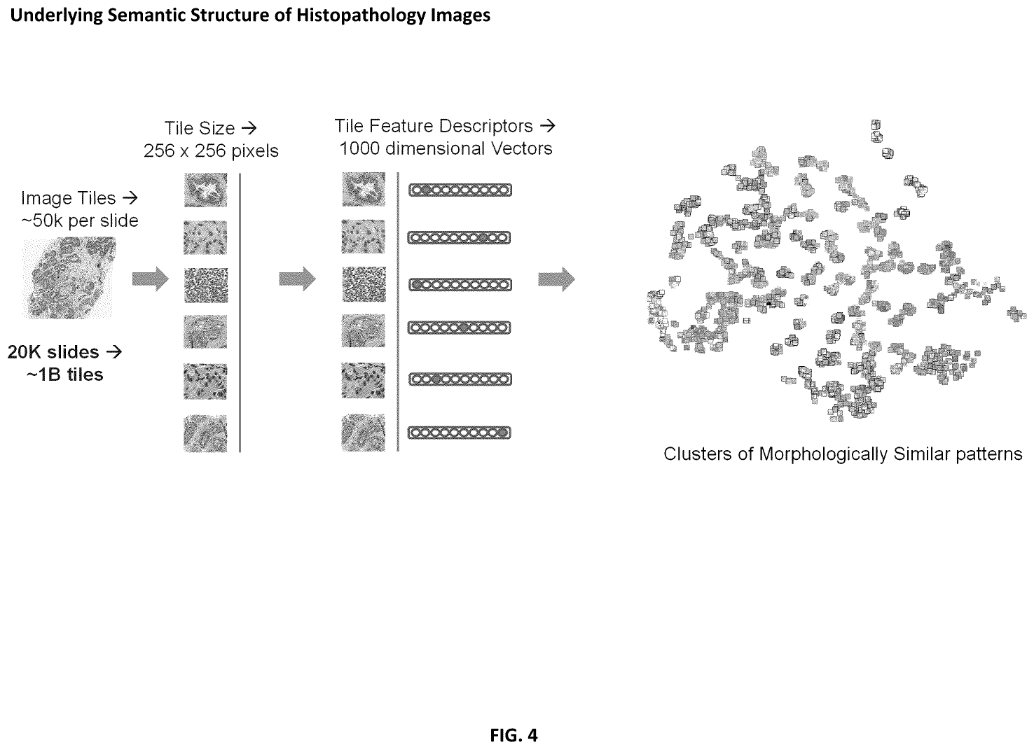

FIG. 4 illustrates an example of an underlying semantic structure of histopathology images. The system may segment image tiles into sub-tiles of a predetermined size, such as 256×256 pixels. Dimensional vectors may be created by processing the sub-tiles.

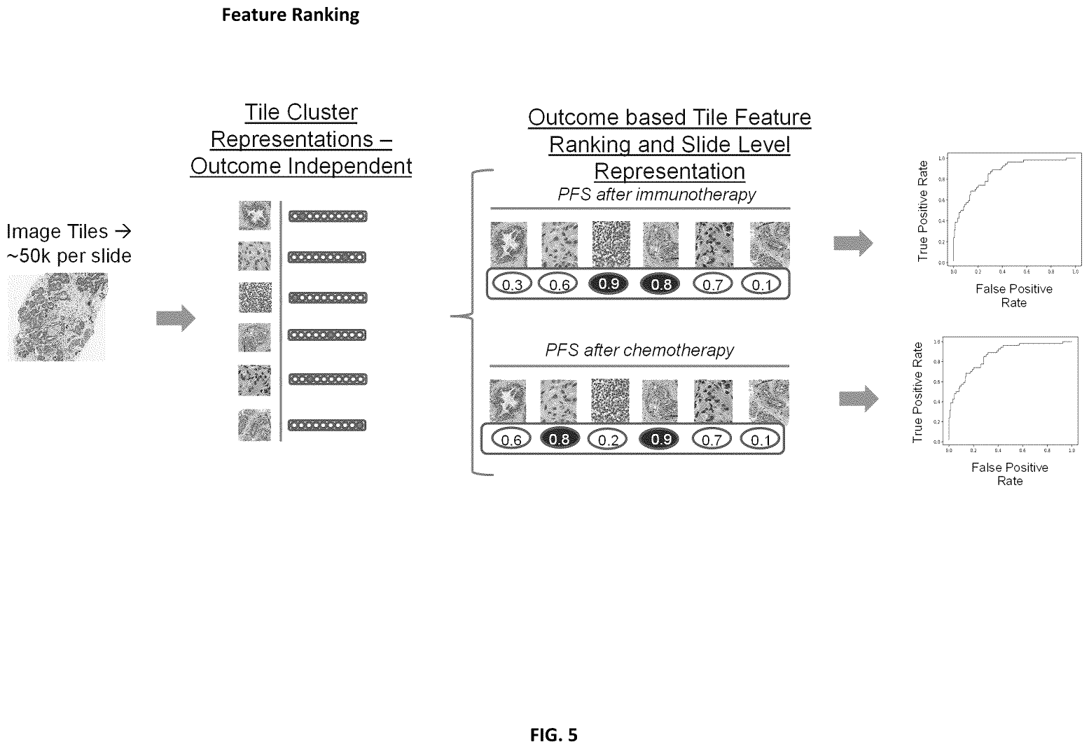

FIG. 5 illustrates an example feature ranking process according to an embodiment. The system may perform feature ranking on the image tiles/sub-tiles and determine a true or false positivity rate. The figure illustrates progression-free survival (PFS) after immunotherapy and PFS after chemotherapy.

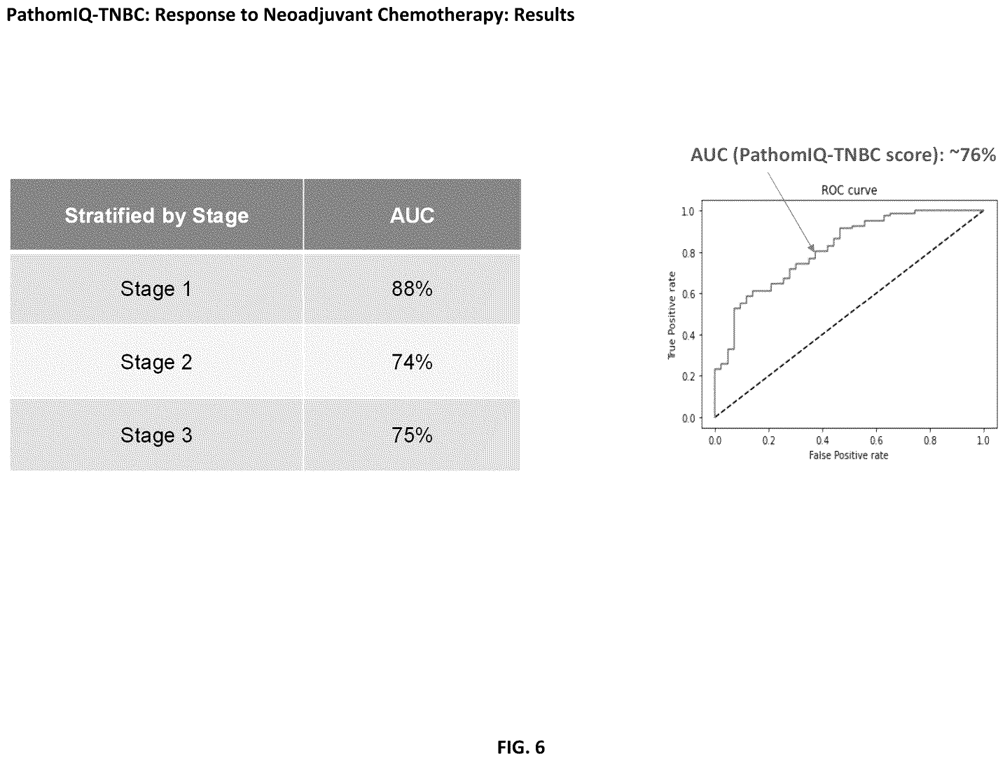

FIG. 6 illustrates a table and graph depicting a response to neoadjuvant chemotherapy. The figure depicts an AUC percentage stratified by stages 1, 2 and 3. An ROC curve depicts an AUC of about 76%.

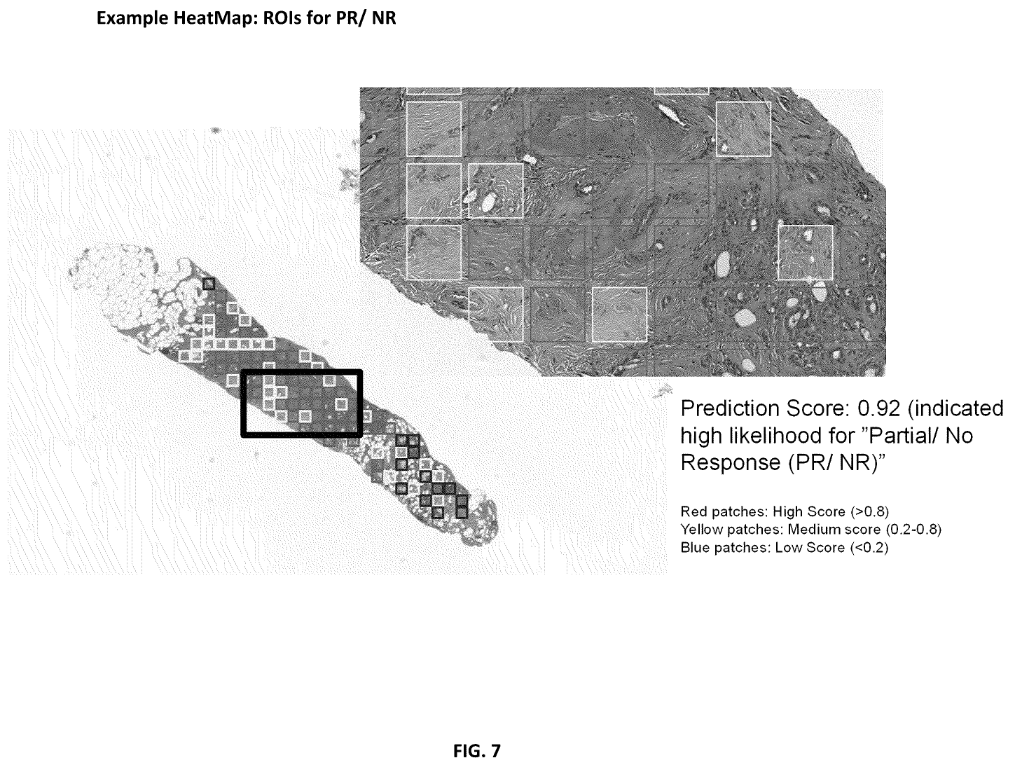

FIG. 7 illustrates an example heatmap relating to ROIs for PR/NR according to an embodiment. The heatmap depicts example results as generated by the system. The heatmap includes patches of a high score (>0.8), patches of a medium score (0.2-0.8), and patches of a low score (<0.2). An example prediction score of 0.92 would indicate a high likelihood for “Partial/response (PR/NR)”.

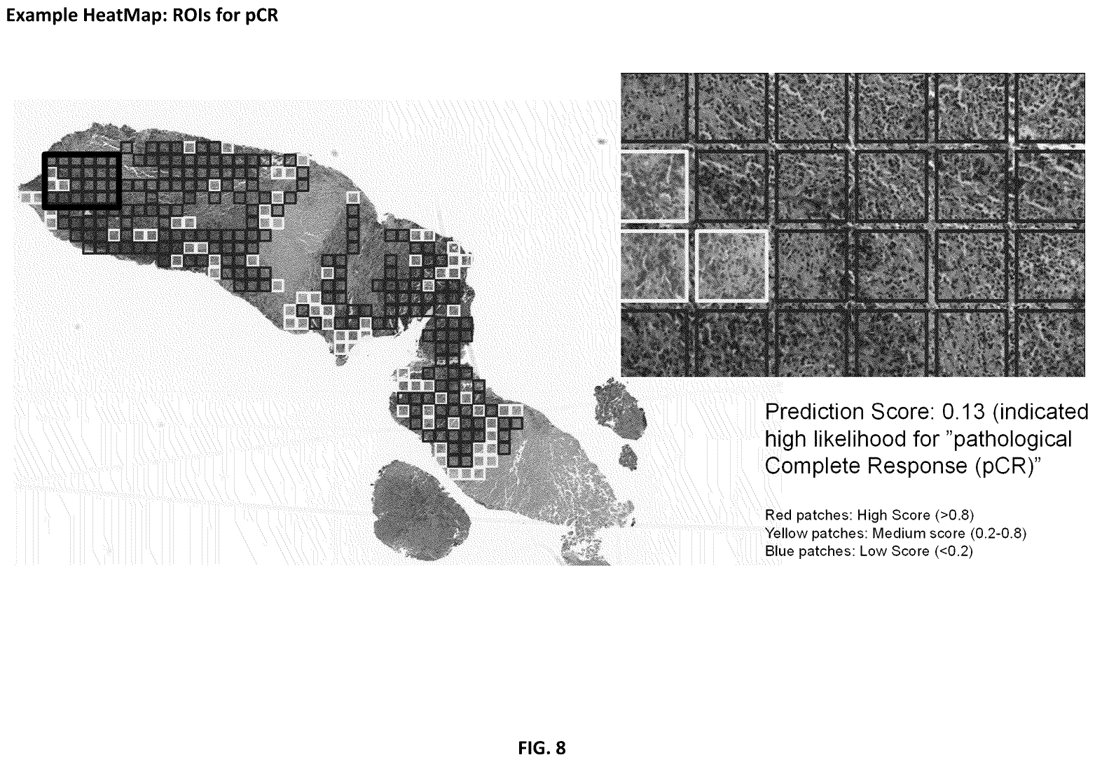

FIG. 8 illustrates an example heatmap relating to ROIs for pCR. The heatmap depicts example results as generated by the system. The heatmap includes patches of a high score (>0.8), patches of a medium score (0.2-0.8), and patches of a low score (<0.2). An example prediction score of 0.13 would indicate a high likelihood for “pathological Complete Response (pCR)”.

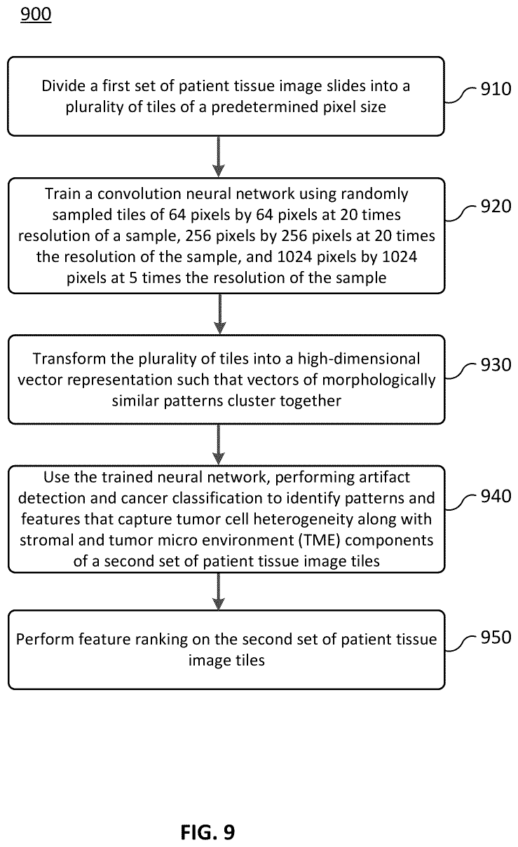

FIG. 9 illustrates a method 900 for predicting patient outcome to neoadjuvant chemotherapy for triple negative breast cancer. In step 910, the system divides a first set of patient tissue image slides into a plurality of tiles of a predetermined pixel size. In step 920, a convolution neural network is trained using randomly sampled tiles of 64 pixels by 64 pixels at 20 times resolution of a sample, 256 pixels by 256 pixels at 20 times the resolution of the sample, and 1024 pixels by 1024 pixels at 5 times the resolution of the sample. In step 930, the system transforms the plurality of tiles into a high-dimensional vector representation such that vectors of morphologically similar patterns cluster together. In step 940, using the trained neural network, the system performs artifact detection and cancer classification to identify patterns and features that capture tumor cell heterogeneity along with stromal and tumor micro environment (TME) components of a second set of patient tissue image tiles. In step 950, the system performs feature ranking on the second set of patient tissue image tiles.

In some embodiments, differential proteogenomic analysis between High Scoring and Low Scoring ROIs may result in discovery of novel proteogenomic biomarkers that drive tumor progression. This approach is unique as it uses disease outcome as the starting point to narrow down to distinct areas of the tissues correlated therapy outcome for proteogenomic interrogation instead of predicting outcome starting from known markers.

### Further Workflow Description:

Input: The model takes as input H & E WSIs and known clinical outcome

Tiling: The slides are divided into tiles of size (256×256 pixels)—the number of tiles vary from 10,000 to 100,000 tiles

Feature extraction module: A multi-scale CNN Feature Extractor is trained using randomly sampled tiles of 64×64 at 20×, 256×256 at 20× and 1024×1024 at 5× resolution to capture nuclear detail, glandular context as well as Tumor Micro Environment (TME) elements. Each tile is transformed to a high-dimensional vector representation, such that vectors of morphologically similar patterns cluster together. The CNN model is trained using millions of examples of varied morphologic features such as benign glands, DCIS, tubular and non-tubular invasive cancer, inflammation, necrosis, fibrosis etc. extracted from the images annotated by expert breast pathologists. The loss function is defined as the cross entropy between predicted probability and the ground-truth.

The vectors are clustered to identify multiple sub-patterns for each label—this generates hundreds of clusters of morphologically similar patterns in high dimensional vector space that do not have any known pathology label, but represent a specific phenotype. This analysis provides information beyond traditional histopathological diagnosis by identifying patterns and features that capture tumor cell heterogeneity along with the stromal and TME components that are not easily distinguished and recognized by human eyes. This enables downstream tasks of ranking these patterns and identifying specific patterns that have high or low prognostic/predictive value.

Feature ranking module: A score is assigned to each image tile cluster based on its correlation with patient outcome. A low score (around 0) represents the tile that will show up mainly in patients that achieve pCR, while a high score (around 1) represents failure to achieve pCR. These scores are generated using a set of weights that are learnt from known patient outcome obtained retrospectively. The tile level scores are combined to a slide level morphometric score to predict patient disease outcome using weights, which are determined by a neural network.

Outcome prediction: The morphology scores are combined with known clinical features such as clinical TNM staging to come up with a combined classifier for patient outcome prediction.

Test: The trained model is used to predicted on a blinded set of previously unseen cases to determine the model accuracy, resulting in 70-90% accuracy across subsets of the data.

In some embodiments, digital whole-slide images (WSI) of hematoxylin and eosin—stained core biopsies of 165 (pathologic complete response (pCR) in 60 and non-pCR in 105) and 78 (pCR in 31 and non-pCR in 47) TNBC patients were used for training and validation of the model. The CNN based AI model was trained to extract morphometric features at visual and sub-visual levels from the WSIs in an unsupervised way. It transformed image tiles from WSIs into high dimensional vector representations that generated multiple clusters of morphologically similar patterns. Downstream ranking of these clusters using neural networks led to the identification of Regions of Interest (ROI) that had high or low predictive value for NAC response. Patch level morphometric scores (MS) assigned to the ROIs combined with clinical tumor, node and metastasis (TNM) stage gave final AI prediction scores; a low score close to 0 and a high score close to 1 represented a high or low probability of response, respectively.

The predictive ability of the model was evaluated using receiver operating characteristic (ROC) analysis which showed area under the curve of 75.5% for the entire cohort and 88.1%, 73.7%, and 74.7% for patients with stage I, II, and III disease, respectively.

### Patient Evaluation

We selected patients diagnosed with TNBC under an institutional review board—approved research protocol with waiver of informed patient consent. The diagnosis of TNBC was based on conventional histopathological examination of H & E-stained tissue sections of formalin-fixed and paraffin-embedded tissue blocks from ultrasound-guided core needle biopsies, including the results of ancillary immunohistochemical staining for ER, PR, and HER2 with or without fluorescence in situ hybridization testing for evaluation of HER2 gene amplification status in selected patients. TNBC diagnosis was established based on ASCO/CAP guidelines with documentation of the presence of invasive tumor in H & E sections together with lack of expression for ER, PR, and HER2 proteins on immunohistochemical staining and negative findings for HER2 gene amplification. All patients included in the retrospective study received NAC that included anthracycline and taxanes at our institution and underwent surgical resection after completion of NAC, consisting of partial or complete mastectomy along with sentinel node biopsy or either targeted or complete axillary lymph node dissection. The surgical resection specimen slices, were radiographed, and the tumor bed was thoroughly sampled to establish the presence or absence of residual tumor. The patients' demographics, initial clinical stage, and final pathologic tumor stage based on the American Joint Committee on Cancer staging system, eighth edition along with extensive clinico-pathological, treatment and outcomes data were recorded. A pCR to NAC was defined as no residual invasive tumor in the breast and no evidence of metastatic tumor in the lymph nodes and thus included pathologic T categories TO or Tis and N categories N0 or N(i+).

The H & E glass slides of the pre-chemotherapy core needle biopsy performed at the time of initial diagnosis from the 243 patients were scanned using a Leica/Aperio AT2 scanner at ×20 magnification to create WSIs. The 243 WSIs were divided to training (165) and test (78) sets ensuring equitable distribution based on patient demographics, clinical, pathological features and response to NAC.

Development of the AI prediction model entailed four essential steps for building the prediction scores of individual patients to predict response to NAC. These steps included input, tiling, feature extraction, and feature ranking. The WSIs were divided into tiles of 256×256 pixels. The number of tiles varied from 10,000 to 100,000 in the core biopsy images. The feature extraction module entailed the building of a multi-scale CNN feature extractor for capturing subvisual features in the images. We first trained the model using WSIs of H & E-stained tissue sections of invasive mammary carcinoma from the National Institute of Health Cancer Genomic Atlas Breast Adenocarcinoma (TCGA-BRCA) data set. The data set contained 799 H & E WSIs including core needle biopsies as well as surgical specimens belonging to all genomic types. Areas of tumor, necrosis, stroma and TILs within stroma, hemorrhage/blood, adipose tissue as well as benign breast glands were annotated by expert pathologists and were used to train a multi-scale CNN feature extractor. This training enabled the model to learn the variations in tumor and microenvironment landscape of breast cancer in an outcome independent manner. The CNN feature extractor was trained using randomly sampled tiles of 64×64 at 20×, 256×256 at 20×, and 1024×1024 at 5× resolution to capture nuclear detail, glandular differentiation, and TME elements. The trained CNN feature extractor was fed as input the WSIs of H & E sections of the pre-chemotherapy core biopsy specimens from patients with known clinical outcomes. Each tile was converted to a 1024-dimensional vector representation, such that vectors of morphologically similar patterns clustered together.

Loss of function was defined as the cross entropy between predicted probability and the ground-truth.

The vectors were clustered to identify multiple sub-patterns for each label—this generated many clusters that exhibited morphologically similar patterns but had no known pathology correlate and therefore resulted in the identification of specific phenotypes. This analysis provided information beyond traditional histopathological diagnosis by identifying patterns and features that capture tumor cell heterogeneity along with the stromal and TME components, including spatial localization of TILs The extraction of the patterns enabled downstream ranking and identification of specific patterns that have high or low predictive value for ascertaining response to NAC.

The final step in developing the AI-based prediction system involved the creation of a feature ranking module. A score was assigned to each image tile cluster based on its association with patient response to NAC. A low score (around 0) represented association with pCR, while a high score (around 1) represented failure to achieve pCR. These scores were generated using a set of weights that were learned from known patient outcomes in the training set. The tile-level scores were combined with a slide-level morphometric score for to obtain the final morphology score for each individual TNBC patient. To train the feature ranking model, we used the attention mechanism, which uses a weighted average of tiles (low dimensional embeddings) where weights are determined by a neural network. The morphology scores were combined with known clinical features such as the clinical TNM stage to obtain the final AI prediction model which could be used in the testing cohort for predicting response to NAC.

FIG. 22 illustrates and example of tiling an invasive tumor, stroma, necrosis and stromal tumor infiltrating lymphocytes. The figures shows the various tumor components, including morphological features, TILs, stroma, and necrosis, that were used to label the tumors for building the AI prediction model. The figures illustrates tiling of an invasive tumor (A), stroma (B), necrosis (C), and stromal tumor infiltrating lymphocytes (D) using whole-slide digital images of hematoxylin and eosin stained tissue sections of a core biopsy of triple-negative breast cancer. This tiling was used for feature extraction and ranking to build the deep convolutional neural network-based artificial intelligence model to predict response of TNBC patients to neoadjuvant chemotherapy

### Statistical Analysis

Receiver operating characteristic (ROC) analysis was used to evaluate the predictive ability of the AI-based prediction system. Higher scores were associated with a lower probability of response to NAC. Performance of the AI prediction model was summarized by the area under the curve (AUC). The AUC was compared with a null value of 0.60 using a one-sided test with a 5% significance level. This comparison was made by taking bootstrap samples of the AUC and comparing those to the null rate of 0.60. To further illustrate the predictive power of the model at the highest and lowest scores, the patients were divided into quartiles based on the score data. Patients in the top quartile of scores were predicted to be non-responders, and those in the bottom quartile of scores were predicted to be complete responders. The positive predictive value was computed in the first group and the negative predictive value in the second group. In addition, the predictive ability of the model in patients with different clinical disease stages was also evaluated using ROC analysis. All statistical analyses were performed using the R version 3.6.1 and a significance level of 5%. No adjustments for multiple testing were made due to the exploratory nature of the study.

### Results

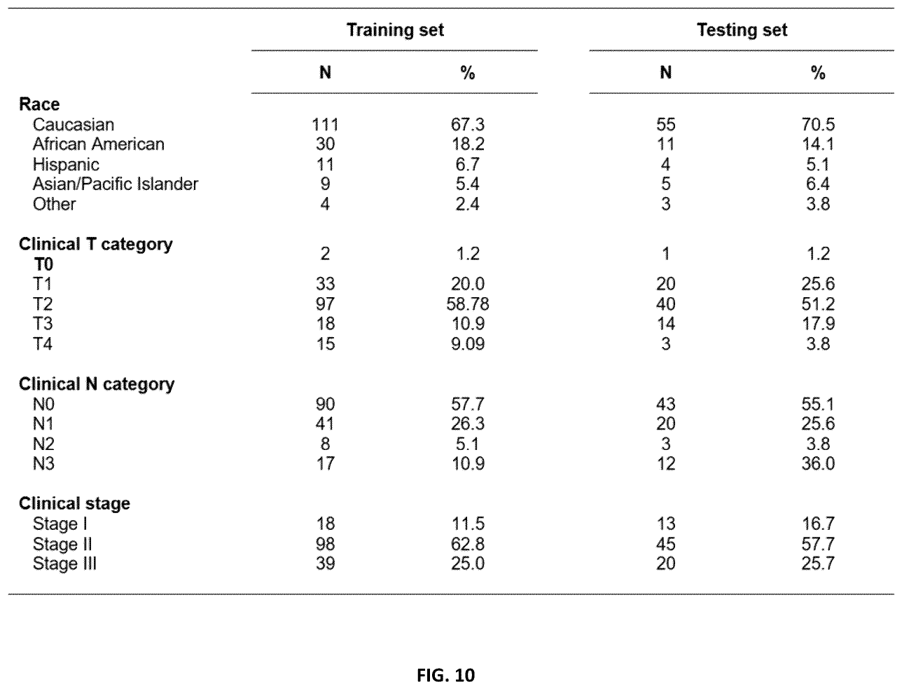

FIG. 10 illustrates a table depicting patient demographics and characteristics. The demographic and clinical characteristics of the patients in the training and test sets are summarized in the table. Characteristics of the training and testing cohorts included in the development of the deep convolutional neural network-based AI model to predict response to neoadjuvant chemotherapy of triple negative breast cancer patients is depicted.

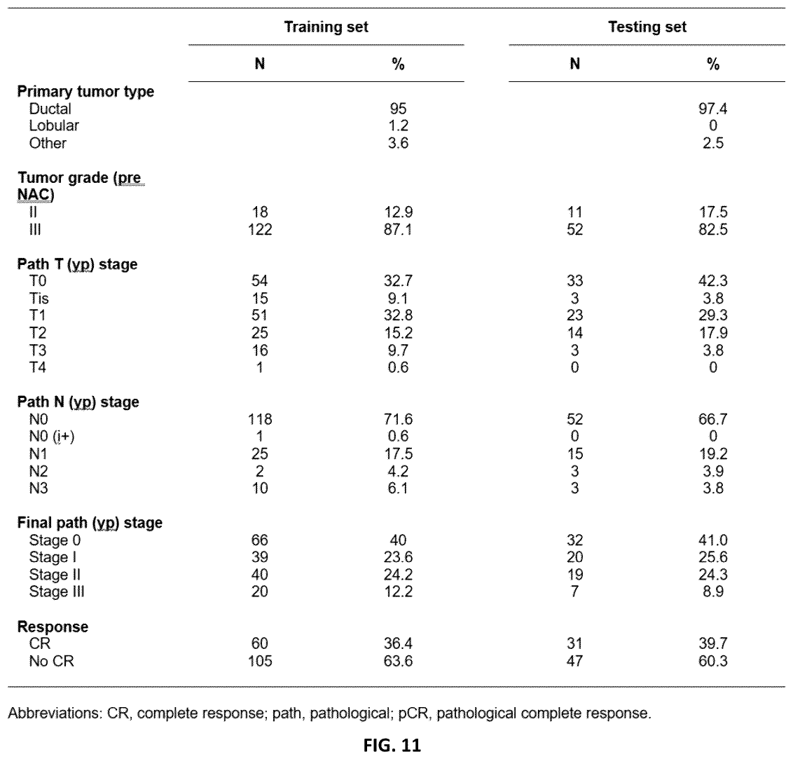

FIG. 11 illustrates a table depicting pathological findings. The pathological findings from the training and test cohort are summarized in the table. Pathological features of the training and testing cohorts included in the development of the deep convolutional neural network-based AI model to predict response to neoadjuvant chemotherapy of triple-negative breast cancer is depicted.

### AI Score of Testing Cohort Tumors

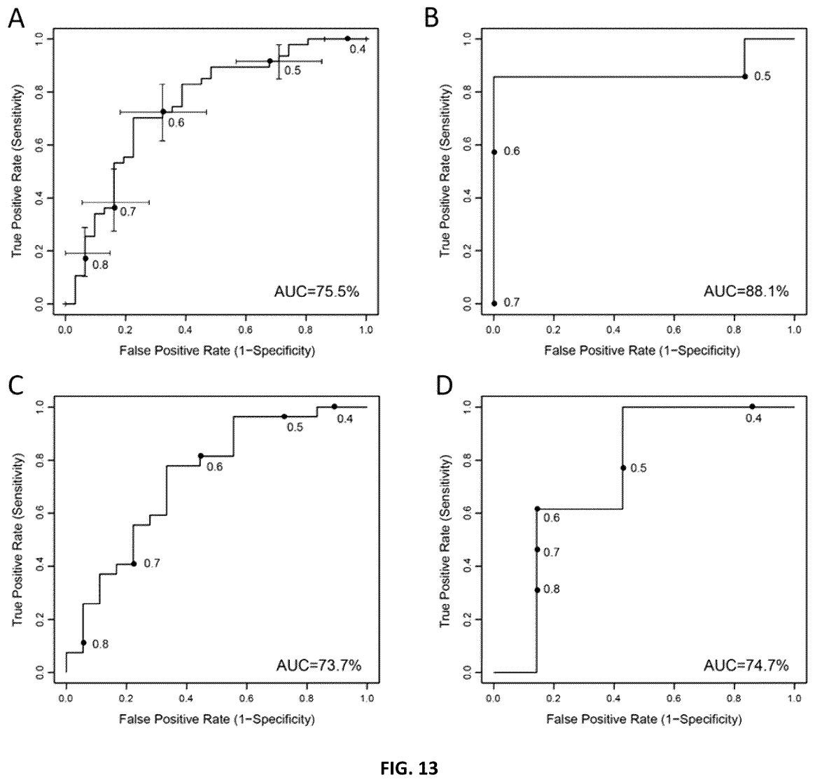

FIG. 13 illustrates exemplary AI prediction scores for four charts A, B, C and D. The y-axis identifies the true positive rate (sensitivity) and the x-axis depicts the false positive rate (1—specificity). The AI prediction scores of the 78 patients in the testing cohort ranged from 0 to 1.0. The predictive ability of the AI score in the entire testing cohort ascertained by ROC analysis demonstrated an AUC of 75.5% (FIG. 2A). This AUC was significantly higher than the protocol-specified null hypothesis of 0.60 (p=0.016).

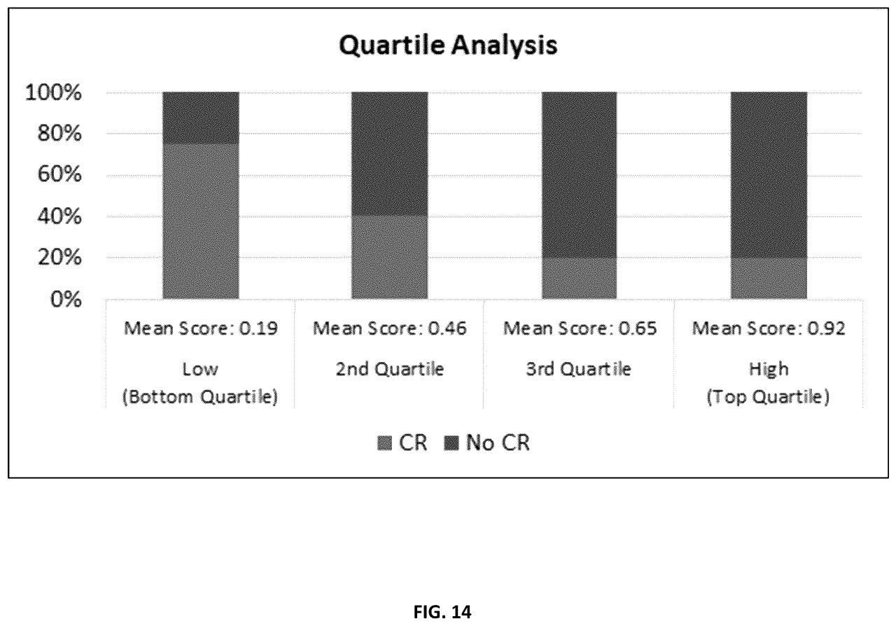

FIG. 14 illustrates an exemplary graph depicting quartile analysis. The performance of the AI scores were also analyzed based on their distribution into quartiles. The deep convolutional neural network-based artificial intelligence (AI) prediction scores of the AI model divided into quartiles and response to neoadjuvant chemotherapy for the 78 triple-negative breast cancer patients in the testing cohort are depicted. Patients in the top quartile with the highest scores were predicted to not have a pCR, and those in the bottom quartile with the lowest scores were predicted to have a pCR. Of the 20 patients in the lowest score quartile, 15 experienced pCR, yielding a positive predictive value for pCR of 75%. Of the 20 patients in the highest score quartile, 16 did not have pCR, yielding a negative predictive value of 80%.

The performance characteristics of the AI score was also estimated across the clinical stages of the TNBC patients. Of the 13 patients with stage I disease, 6 experienced pCR and 7 did not. Of the 45 patients with stage II disease, 18 experienced pCR and 27 did not. Of the 20 patients with stage III disease, 7 experienced pCR and 13 did not. The AUC in the ROC analysis for patients with stage I, II, and III disease was 88.1%, 73.7%, and 74.7%, respectively.

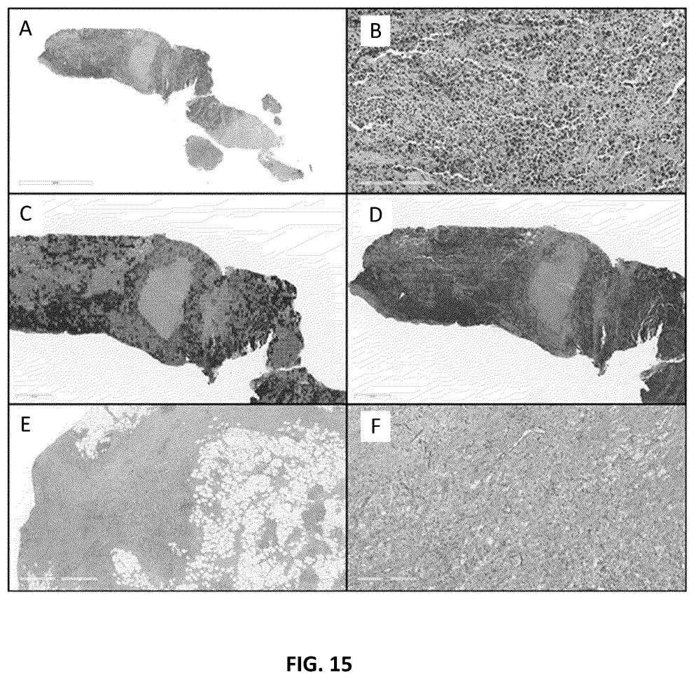

FIG. 15 illustrates exemplary images from pre-chemotherapy biopsy and final surgical pathology. The images depict pre-chemotherapy biopsy and final surgical pathology obtained from a patient with an AI prediction score of 0.13. Hematoxylin and eosin (H & E) stained core biopsy sample (A) of a triple-negative breast cancer procured before neoadjuvant chemotherapy showing high Nottingham histologic grade and infiltration of tumor-infiltrating lymphocytes (TILs) (B). The tiling and labeling of a whole-slide digital image of the H & E section of the core biopsy to include features of the tumor, stroma, and TILs to obtain the final score is shown in panels (C) and (D). The deep convolutional neural network-based artificial intelligence prediction score of this case was 0.13, indicating a high likelihood of response. The prediction of probability of response to neoadjuvant chemotherapy was accurate as evidenced by complete response to neoadjuvant chemotherapy. The tumor bed with no residual tumor is shown in panels (E) and (F).

### Further Discussion

The development of our AI-based system using a CNN built on features extracted from WSIs of TNBC demonstrates that this approach can serve as a powerful tool for predicting response to NAC in this patient population. The predictive power of the AI-based system was highest in patients with stage I disease at 88.1% compared with 74% and 75%, respectively, in those with stage II and stage III disease. This novel AI-based prediction system extracts the otherwise undiscernible information in H & E-stained tissue sections of the invasive tumor, basically integrating the global genomic and proteomic changes manifested in the morphological appearance of the tumor. The exploitation of sub visual collective features of the tumor and TME in H & E-stained tissue sections for building an AI-based system to predict response to NAC in patients with TNBC has not been described previously.

Traditional supervised AI approaches have focused on applications such as tumor detection, estimation of TILs, and quantification of known biomarkers such as PD-L1 immuno expression in malignant tumors. The accuracy of these models is limited to the predictive power of features under investigation that are already known based on evaluations using conventional methodologies. The major contribution of such models is automation and objective evaluation that overcomes inter-observer variability. In contrast, our DL and CNN-based AI approach facilitates the discovery of novel and previously unrecognized morphological features that drive tumor progression and influence therapeutic response. Our model converts image patches into mathematical vector representations to generate hundreds of clusters of morphologically similar patterns using a state-of-the-art deep CNN. It then ranks these image clusters from the WSIs to identify novel regions of interest (ROIs) which have high or low prognostic/predictive values associated with patient outcome information. These ROIs are used to generate a morphometric score to predict response to NAC. It is to be noted that the ROIs capture both cancer and TME landscapes, including spatial distribution of the immune and stromal cells relative to the cancer zones that play major roles in tumor progression and resistance to therapy. The ability to incorporate not only the extent of TILs but also their spatial distribution is a distinct advantage of our AI model for predicting the response to NAC in TNBC patients. There are reports using CNN for evaluation of TILs' spatial organization, which by itself was found to be prognostic for outcomes of different types of malignant tumors. Yuan et al developed a model to investigate the spatial distribution of TILs and tumor cells in TNBC. The model identified three categories of TILs according to their proximity to tumor cells.

The potential of an AI model using deep CNN to extract nuclear features alone for predicting response to NAC was recently reported by Dodington et al. (33). These authors used a small cohort of 20 TNBC and 30 HER2-positive breast cancer patients to evaluate their AI model based on multiple deep CNNs utilizing nuclear and image characteristics. The model could successfully classify 79% of cases, including 89% of pCRs and 62% of pathological partial responses. Romo-Bucheli et al used a CNN-based model to automatically detect mitotic figures in WSIs of ER-positive breast tumors. The distribution of mitotic figures was significantly different between patients with high compared with low risk of breast cancer recurrence as determined by Oncotype DX. Unlike these reports, our study goes way beyond to leverage a deep CNN approach for unsupervised extraction of morphometric features exhibited in H & E slides to gather collective information of the tumor, stroma and tumor microenvironment and correlate the extracted overall features with response to NAC in TNBC.

Several potential indicators using relevant clinical, radiologic, and histopathologic features and genomic signatures have been proposed for predicting response to NAC. The histologic grade of TNBC, extent of TILs, and proliferation index of the invasive tumor based on labeling for MIB1 have all been considered for prediction of response. Several genomic investigations have proposed transcriptomic signatures related to proliferation- and immune-related genes, stroma-related genes, and overall transcriptomic profiles that can be correlated with response to NAC. Recently, the promising potential of a novel three-gene score based on the cell proliferation—related E2F gene was reported as a promising prognostic and predictive biomarker of response to NAC in patients with TNBC. A recent study reported that a predictive model using routinely collected clinical and demographic variables was able to predict pCR for NAC in a mixed dataset of ER/PR+. HER2+ and TNBC cases with 0.70 AUC. Although some of these proposed predictors are promising, none has yet made an impact in the standard-of-care management of TNBC with reproducible and reliable performance of high sensitivity and specificity. The search for identifying predictors of response to NAC that can be translated to standard-of-care clinical practice is ongoing and is an unmet challenge in TNBC.

The need for predictors of response to NAC in TNBC is well recognized, and the search for robust predictors has been ongoing for years. Unlike the pursuit of morphological, genomic, and proteomic biomarkers of response individually, the AI approach using deep CNN described herein provides the opportunity to gather global morphometric alterations in the tissue that can be effectively exploited to predict response to NAC. Our AI prediction model allowed us to establish scores based on ranking several features incorporated in the deep CNN. High AI scores obtained from the H & E slides of TNBC patients predicted lack of response to NAC in 80.7%, and low scores predicted response in 69.2%. The model's performance was best in patients with early-stage TNBC. The AI-based prediction system has the potential for use in the clinical management of patients with TNBC, particularly those with stage I disease. The possibility of de-escalating chemotherapy in early-stage TNBC patients who may respond favorably to NAC is of great interest. Eliminating anthracyclines in the NAC regimen and avoiding immunotherapy in patients who will most likely be exceptional responders to NAC are some considerations for modifying the treatment of these patients. On the other hand, the possibility of altering the chemotherapeutic regimen of patients who are deemed to be resistant to conventional NAC—including enrollment in clinical trials using experimental therapeutic agents—may also be desirable. The availability of AI-based prediction systems that can inform the clinician regarding the possibility of response or resistance to NAC at the time of initial diagnosis may have tremendous clinical utility in the management of TNBC.

The AI prediction model can be improved further with incorporation of additional features, including pertinent genomic and proteomic biomarkers. In addition, the results of emerging novel tissue imaging modalities, such as infrared chemical and molecular imaging, can most likely improve the contribution of the sub visual morphometric features used to build the AI-based prediction system. Infrared spectroscopic imaging can provide detailed information of several key proteomic, lipidomic, and genomic constituents in the tissues which may be useful for incorporation into the AI prediction model. Future refinement of the model with the input of potential genomic and proteomic biomarkers is envisioned to increase the model's performance beyond what was achieved in this first study. A unique advantage of the AI approach includes their role in facilitating the performance of focused proteogenomic evaluation of selected areas of the tissue section with distinctly different morphometric scores. Differential proteogenomic analysis of the tissue between high and low scoring ROIs with known outcomes may result in the discovery of novel proteogenomic biomarkers that drive tumor progression. Such an approach was recently used to predict early recurrence of prostate carcinoma following radical prostatectomy and included the discovery of a new potential biomarker that was identified following genomic and proteomic evaluation of the tumor areas associated with high morphometric scores. The AI-based predictive approach is unique in that it uses disease outcome as the starting point to narrow down downstream genomic and proteomic investigations to distinct areas of the tissues rather than initiating the investigation using the entire tissue for exploration of global changes or by using known markers for predicting outcome.

We demonstrate the performance of a novel AI prediction model based on CNN using a training and a testing cohort of TNBC patients with known response to NAC. Although the sample size of the testing cohort was limited in this first report, the results are promising and show potential for guiding clinical management of TNBC patients and warrant further validation and the simplicity of scanned slides as the starting point makes this effort highly scalable. Improvements to the model, including validation in a larger cohort of TNBC patients and incorporation of additional biomarkers to the model, are underway. Our study exemplifies the utility of WSIs for building CNN-based AI prediction models for TNBC patients that have the potential to function as robust ancillary digital pathology tools in conjunction with standard-of-care pathologic evaluation of malignant tumors.

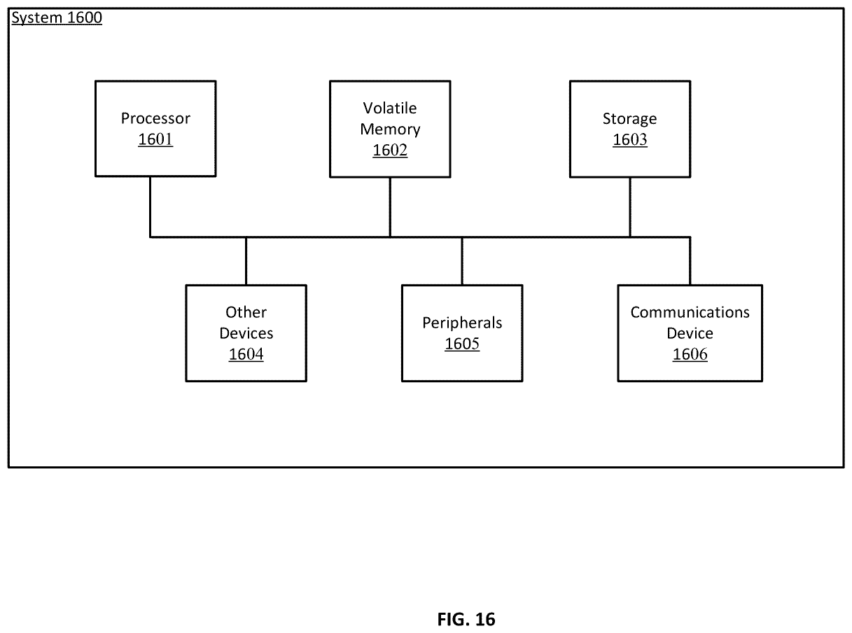

FIG. 16 is a diagram illustrating an exemplary computer that may perform processing in some embodiments. Exemplary computer 1600 may perform operations consistent with some embodiments. The architecture of computer 1600 is exemplary. Computers can be implemented in a variety of other ways. A wide variety of computers can be used in accordance with the embodiments herein.

Processor 1601 may perform computing functions such as running computer programs. The volatile memory 1602 may provide temporary storage of data for the processor 1601. RAM is one kind of volatile memory. Volatile memory typically requires power to maintain its stored information. Storage 1603 provides computer storage for data, instructions, and/or arbitrary information. Non-volatile memory, which can preserve data even when not powered and including disks and flash memory, is an example of storage. Storage 1603 may be organized as a file system, database, or in other ways. Data, instructions, and information may be loaded from storage 1603 into volatile memory 1602 for processing by the processor 1601.

The computer 1600 may include peripherals 1605. Peripherals 1605 may include input peripherals such as a keyboard, mouse, trackball, video camera, microphone, and other input devices. Peripherals 1605 may also include output devices such as a display. Peripherals 1605 may include removable media devices such as CD-R and DVD-R recorders/players. Communications device 1606 may connect the computer 1600 to an external medium. For example, communications device 2606 may take the form of a network adapter that provides communications to a network. A computer 1600 may also include a variety of other devices 1604. The various components of the computer 1600 may be connected by a connection medium such as a bus, crossbar, or network.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general-purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any programming language. It will be appreciated that a variety of programming languages may be used to implement the teachings of the disclosure as described herein.

What is claimed is:

1. A method for predicting patient outcome to neoadjuvant chemotherapy for triple negative breast cancer, comprising:
dividing a first set of patient tissue image slides into a plurality of tiles of a predetermined pixel size;
training a convolution neural network using randomly sampled tiles of 64 pixels by 64 pixels at 20 times resolution of a sample, 256 pixels by 256 pixels at 20 times the resolution of the sample, and 1024 pixels by 1024 pixels at 5 times the resolution of the sample;
transforming the plurality of tiles into a high-dimensional vector representation such that vectors of morphologically similar patterns cluster together;
using the trained convolutional neural network to perform artifact detection and cancer classification to identify patterns and features that capture tumor cell heterogeneity along with stromal and tumor micro environment (TME) components of a second set of patient tissue image tiles; and
performing feature ranking on the second set of patient tissue image tiles.

2. The method of claim 1, wherein the predetermined pixel size is 256 pixels by 256 pixels, and where the number of tiles ranges from 10,000 to 100,000 tiles.

3. The method of claim 1, further comprising:
defining a loss function as cross entropy between predicted probability and a ground-truth.

4. The method of claim 1, wherein performing artifact detection and cancer classification comprises:
clustering vectors to identify multiple sub-patterns for a labeled pathology.

5. The method of claim 1, wherein performing feature ranking on the second set of patient tissue image tiles comprises:
performing feature ranking by assigning a tile score to each image tile cluster based on its correlation with a patient outcome, wherein a lower score value on a numeric range represents that the tile will mainly show up in patients that achieve pCR, while a higher score value represents failure to achieve pCR.

6. The method of claim 5, wherein a score is generated using a set of weights that are associated with a known patient outcome obtained retrospectively.

7. The method of claim 6, further comprising:
combining the tile scores to a slide level morphometric score to predict patient disease outcome using weights, which are determined by the neural network.

8. The method of claim 7, further comprising:
combining the slide level morphometric score with known clinical features to determine a combined classifier for patient outcome prediction.

9. The method of claim 1, wherein the convolutional neural network is trained to predict response of triple negative breast carcinoma to neoadjuvant chemotherapy.

10. The method of claim 9, further comprising the operation of:
training the convolutional neural network with data of histopathological components together with clinical features including pre-chemotherapy clinical tumor, node, metastasis (TNM) stage and post neoadjuvant chemotherapy pathologic tumor, necrosis and metastasis stage (ypTNM).

11. The system of claim 1, wherein the first set of patient tissue image tiles include histopathological features in hematoxylin and eosin-stained tissue sections of whole slide digital images of pre-chemotherapy core biopsies of triple negative breast carcinoma.

12. The system of claim 11, wherein the patient tissue image tiles include annotated components of at least one or more of tumor, stroma, tumor infiltrating lymphocytes, hemorrhage, necrosis, and/or a combination thereof.

13. A system for predicting patient outcome to neoadjuvant chemotherapy for triple negative breast cancer, the system comprising one or more processors configured to perform the operations of:
dividing a first set of patient tissue image slides into a plurality of tiles of a predetermined pixel size;
training a convolution neural network using randomly sampled tiles of 64 pixels by 64 pixels at 20 times resolution of a sample, 256 pixels by 256 pixels at 20 times the resolution of the sample, and 1024 pixels by 1024 pixels at 5 times the resolution of the sample;
transforming the plurality of tiles into a high-dimensional vector representation such that vectors of morphologically similar patterns cluster together;
using the trained neural network to perform artifact detection and cancer classification to identify patterns and features that capture tumor cell heterogeneity along with stromal and tumor micro environment (TME) components of a second set of patient tissue image tiles; and
performing feature ranking on the second set of patient tissue image tiles.

14. The system of claim 9, wherein the predetermined pixel size is 256 pixels by 256 pixels, and where the number of tiles ranges from 10,000 to 100,000 tiles.

15. The system of claim 9, further comprising the operations of:
defining a loss function as cross entropy between predicted probability and a ground-truth.

16. The system of claim 9, wherein performing artifact detection and cancer classification comprises:
clustering vectors to identify multiple sub-patterns for a labeled pathology.

17. The system of claim 9, wherein performing feature ranking on the second set of patient tissue image tiles comprises:
performing feature ranking by assigning a tile score to each image tile cluster based on its correlation with a patient outcome, wherein a lower score value on a numeric range represents that the tile will mainly show up in patients that achieve pCR, while a higher score value represents failure to achieve pCR.

18. The system of claim 13, wherein a score is generated using a set of weights that are associated with a known patient outcome obtained retrospectively.

19. The system of claim 14, further comprising the operations of:
combining the tile scores to a slide level morphometric score to predict patient disease outcome using weights, which are determined by the neural network.

20. The system of claim 15, further comprising the operations of:
combining the slide level morphometric score with known clinical features to determine a combined classifier for patient outcome prediction.

21. The system of claim 9, wherein the convolutional neural network is trained to predict response of triple negative breast carcinoma to neoadjuvant chemotherapy.

22. The system of claim 17, further comprising the operation of:
training the convolutional neural network with data of histopathological components together with clinical features including pre-chemotherapy clinical tumor, node, metastasis (TNM) stage and post neoadjuvant chemotherapy pathologic tumor, necrosis and metastasis stage (ypTNM).

23. The system of claim 9, wherein the first set of patient tissue image tiles include histopathological features in hematoxylin and eosin-stained tissue sections of whole slide digital images of pre-chemotherapy core biopsies of triple negative breast carcinoma.

24. The system of claim 18, wherein the patient tissue image tiles include annotated components of at least one or more of tumor, stroma, tumor infiltrating lymphocytes, hemorrhage, necrosis, and/or a combination thereof.
